(19) United States
(12) Patent Application Publication
(10) Pub. No.: US 2010/0223070 A1
(43) Pub. Date: Sep. 2, 2010

(54) APPARATUS AND METHOD FOR MORNITORING HEALTHCARE DATA

(75) Inventors: Declan Patrick Kelly, Shanghai (CN); Sheng Jin, Shanghai (CN)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., Eindhoven (NL)

(21) Appl. No.: 12/161,702

(22) PCT Filed: Jan. 15, 2007
(86) PCT No.: PCT/IB2007/050108

§ 371 Date: Jul. 22, 2008

(30) Foreign Application Priority Data
Jan. 27, 2006 (CN) 200610004733.8

(51) Int. Cl. G06Q 50/00; A61B 5/00; G06F 17/30

(52) U.S. Cl. 705/3; 600/300; 707/781; 707/E17.005

(57) ABSTRACT

The present invention provides an interface for monitoring healthcare data at the caregivers' side, the interface comprises receiving means for receiving a part of the healthcare data and an indication(s) of the blocking status of the rest of the healthcare data, presenting means for presenting the healthcare data, and indicating means for indicating the blocking status of the rest of the healthcare data according to the indication. In this way, a better balance between the benefits of the monitored person and that of the caregivers could be reached.

### FIELD OF THE INVENTION

This invention relates generally to the technology of monitoring healthcare data, and more particularly to off-site monitoring healthcare data.

### BACKGROUND OF THE INVENTION

Recently more and more technologies have been proposed for monitoring personal healthcare data, and many of them are off-site monitoring, for example, a doctor at a hospital to a patient at home, a son at his office to an elderly mother at home and etc. For such monitoring activities there may be all kinds of personnel healthcare data gathered by different monitoring devices, which could be transmitted to the caregivers.

Due to various reasons, the person who is under monitoring may not want all of his/her personal healthcare data to be transmitted to the caregivers. Such reasons may include privacy of the monitored person, capacity of communication channel, affordability and etc.

US patent application with publication number US 2005/0101841 A9 (Inventors: Rosan Kaylor, et al, assignee: Kimberly-Clark Worldwide Inc., Publication Date: May 12, 2005) discloses a healthcare network for sharing information concerning the health of a user with at least one outside source. The network includes a biosensor associated with the user that generates a biosensor signal containing the information, and a personal data control means including receiving means for receiving the biosensor signal, input means for receiving a privacy input form the user, and output means for generating a response signal based on the biosensor signal and privacy input.

### OBJECT AND SUMMARY OF THE INVENTION

While current privacy control technology in healthcare monitoring domain may protect the privacy of the monitored person, there are some possibilities that the caregivers and the monitored person may have some disagreements or different needs on what information should be shared among them, especially when an emergency happens from the caregivers' professional perspective.

There are also some possibilities that the out of date version of the data blocked by the privacy control is still presented to the caregivers, and such data may mislead the caregivers.

But it may not be suitable that such out of date data are just simply be removed from presenting to the caregivers, since some of the out of date data may be still valuable, and its value may depending on how old the data are and other considerations, for example, for the corresponding disease how important the data are.

It is a desire of the invention to provide an improved healthcare monitoring system that could provide better balance between the benefits of the monitored person and that of the caregivers.

The desire is achieved in an apparatus for controlling healthcare data at the monitored person side, the apparatus comprises receiving means for receiving the healthcare data, obtaining means for obtaining an user's input of controlling the dispatch of the healthcare data, and transmitting means for transmitting a part of the healthcare data and an indication(s) of the blocking status of the rest of the healthcare data to a outside source according to the user's input.

The desire is achieved by an interface for monitoring healthcare data at the caregivers' side, the interface comprises receiving means for receiving a part of the healthcare data and an indication(s) of the blocking status of the rest of the healthcare data, presenting means for presenting the healthcare data, and indicating means for indicating the blocking status of the rest of the healthcare data according to the indication.

According to one embodiment of the invention, the interface further requesting means for sending a request for demanding the blocked data from a source of the healthcare data. By acknowledging the block status, the caregivers may initiate a request to ask for the data they need.

According to another embodiment of the invention, the indicating means further for indicating how long the rest of the healthcare data are blocked. By knowing the age of the un-updated data, the caregivers may make a judgment on the value of the un-updated data.

Other objects and attainments together with a fuller understanding of the invention will become apparent and appreciated by referring to the following description and claims in conjunction with the accompanying drawings.

### BRIEF DESCRIPTION OF THE DRAWINGS

The invention is explained in further detail, and by way of examples, with reference to the accompanying drawings wherein:

FIG. 1 is a schematic diagram of a controlling apparatus according to one embodiment of the invention,

FIG. 2 is a flow chart illustrating a healthcare data controlling process according to one embodiment of the invention,

FIG. 3 is a schematic structure diagram of a monitoring interface according to another embodiment of the invention, and

FIG. 4 is a flow chart illustrating a healthcare data monitoring process according to another embodiment of the invention.

FIG. 5 is a schematic layout of the healthcare-data-monitoring interface according to another embodiment of the invention.

Throughout the drawings, the same reference numerals indicate similar or corresponding features or functions.

### DETAILED DESCRIPTION OF THE INVENTION

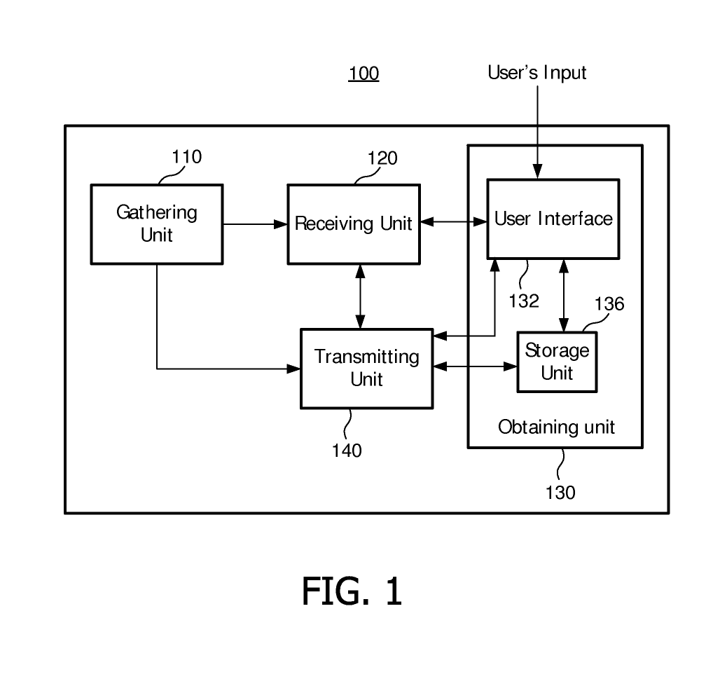

FIG. 1 is a schematic diagram of a controlling apparatus according to one embodiment of the invention. This controlling apparatus could be used at the side of the monitored person.

Controlling apparatus 100 includes a receiving unit 120, an obtaining unit 130 and a transmitting unit 140. Optionally it could further include a gathering unit 110.

The gathering unit 110 gathers the healthcare data. It includes a blood pressure biosensor to gather the blood pressure of the person being monitored. It could also include other types of biosensors gathering other life data, for example, body temperature, heart beat rate, weight, body motions and etc.

The gathering unit 110 also could includes environmental sensors, which gather the life related environmental data, for example, indoor temperature, humidity, and noise level and etc.

The gathering unit could also be other information collecting devices, such as camera and recorder.

The gathering unit could be a complex information-collecting network, which includes all kinds of sensors and other information collecting devices scattered on the body of the person being monitored and the environment this person being monitored is in. This network collects all kinds of life data and/or life related environmental data. And controlling apparatus 100 could act as a gate for this information-collecting network.

Receiving unit 120 receives healthcare data from the gathering unit 110. The healthcare data include life data (body temperature, blood pressure, weight) and life related environmental data (noise level).

If the gathering unit 110 is not integrated in controlling apparatus 100, receiving unit 120 could be any current or future wired/wireless communication receiving device, such as internet connection, intranet connection, and etc, as long as the gathering unit 110 and the receiving unit 120 could build up a communication channel and accomplish data transfer.

Obtaining unit 130 obtains a user's input of controlling the dispatch of the healthcare data, and this user is the person being monitored. Obtaining unit 130 includes a user interface 132, and it may further include a storage unit 136.

The user interface 132 receives the control signals inputted by the user, which classify which part of the healthcare data may be transmitted to the caregivers (blood pressure) and which other part may be blocked form transmitting to the caregivers (body temperature, weight, noise level). The user interface 132 may also present the healthcare data/data structure to the user for his/her classification. Such a classification may be stored in the storage unit 136 for future use.

The storage unit 136 stores and retrieves the user's input of controlling the dispatch of the healthcare. It is a non-volatile flash memory card. It could also be any other storage devices, such as a hard disk, a floppy disc and etc.

This user could also be an authorized representative of the person being monitored. This user may input his/her controlling classification via the user interface 132 and store this controlling classification on the storage unit for future use.

Transmitting unit 140 transmits a part of the healthcare data and an indication(s) of the blocking status of the rest of the healthcare data to a outside source (a monitoring device at the side of caregivers) according to the user input.

There are various ways to integrate the indication into the transmitted data. One example is to add one blocking mark for one blocked data item in the corresponding positions in the file of the whole healthcare data.

Another example is to leave some corresponding data positions blank in the file of the whole healthcare data and to send a separate file to indicate the reasons for the blanks. One reason is that the user blocks the corresponding data. Another reason may be the controlling apparatus could not get the corresponding data that is missing due to the failure of the gathering unit 110. Different reasons may correspond to different indicators; for example, “X” is for blocking (body temperature, weight, noise level), “Y” is for “missing”.

The file transmitted to the caregivers may also include some identification information of the person being monitored.

It is understandable that receiving unit 120 may also could receive some information from the side of the caregivers, for example, a request to override the classification inputted by the user.

It is also understandable that storage unit 136 also could stored the healthcare data as a history record for future retrieval.

The desire of this invention can also be implemented by means of a suitably programmed computer provided with a computer program for controlling healthcare data. The computer program product for controlling healthcare data, comprises code for receiving the healthcare data, code for obtaining an user input of controlling the dispatch of the healthcare data, and code for transmitting a part of the healthcare data and an indication(s) of the blocking status of the rest of the healthcare data to a outside source, according to the user input.

Such a computer program product may be stored in a storage carrier.

These portions of program code may be provided to a processor to produce a machine, such that the code that executes on the processor create means for implementing the functions specified as above.

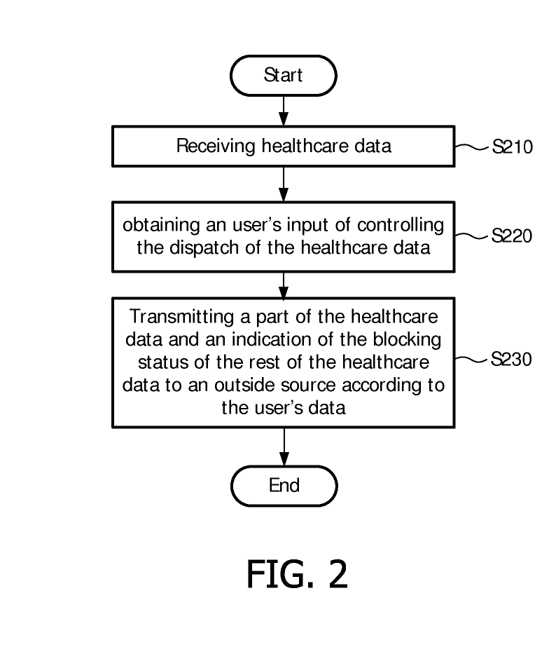

FIG. 2 is a flow chart illustrating a healthcare data controlling process according to one embodiment of the invention. This process could be implemented on controlling apparatus 100.

Firstly, receiving the healthcare data (step S210).

The healthcare data include life data (body temperature, blood pressure, weight) and life related environmental data (noise level).

Secondly, obtaining a user input of controlling the dispatch of the healthcare data (step S220).

The user input classifies which part(s) of the healthcare data (blood pressure) may be transmitted to the caregivers and which other part(s) (body temperature, weight, noise level) may be blocked form transmitting to the caregivers

The user input could be real time acquired via a user interface 132, or it could be stored in a storage unit 136 in advance and be retrieved as needed.

Finally, transmitting a part of the healthcare data and an indication(s) of the blocking status of the rest of the healthcare data to a outside source, according to the user input. (Step S230). The blood pressure data is transmitted to the caregiver side, and the data of the body temperature, weight and noise level are replaced with a mark “X” to indicate the user blocks the data.

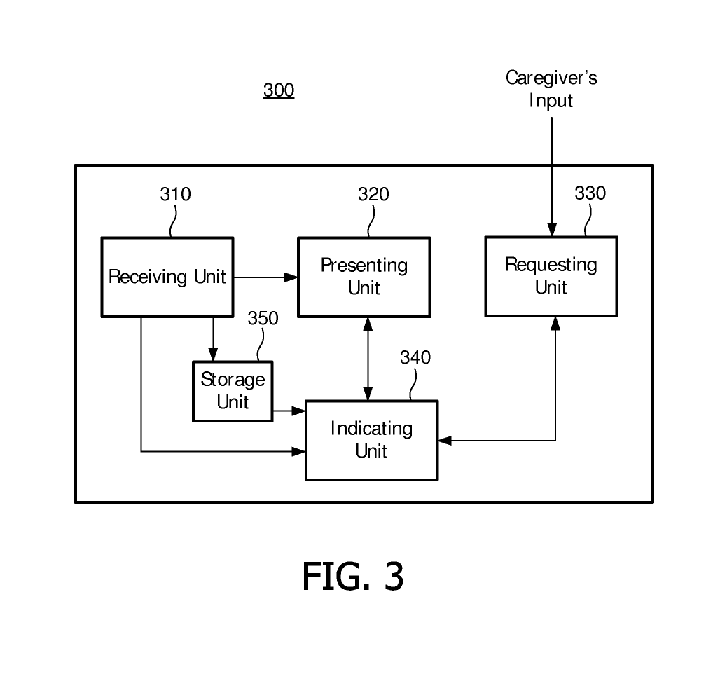

FIG. 3 is a schematic structure diagram of a monitoring interface according to another embodiment of the invention. This monitoring interface could be implemented at the caregivers' side to process the data provided by controlling apparatus 100.

Monitoring interface 300 includes a receiving unit 310, a presenting unit 320, and an indicating unit 340. It could also include a requesting unit 330. And it may further include a storage unit 350.

Receiving unit 310 receives a part of healthcare data (blood pressure) and an indication(s) of the blocking status of the rest of the healthcare data (“X” marks for body temperature, weight and noise level).

Receiving unit 310 could be any current or future wired/wireless communication-receiving device, such as Internet connection, intranet connection, and etc, as long as the controlling apparatus 100 and the monitoring apparatus 300 could build up a communication channel and accomplish data transfer.

Presenting unit 320 presents the healthcare data received by receiving unit 310. In this embodiment, the data of blood pressure is presented by a display. Any other presenting means could also be applied, such as a speaker.

Storage unit 350 stores the data received by receiving unit 310 for future use, and retrieves the stored data as needed. It is a non-volatile flash memory card. It could also be any other storage devices, such as a hard disk, a floppy disc and etc.

Indicating unit 340 indicates the blocking status of the rest of the healthcare data according to the indication. In this embodiment, the received data of body temperature, weight and noise level are “X” marks, accordingly the fields of body temperature, weight and noise level on the display are indicated in a different presenting way from the normal presenting way of presenting unit 320 to show the three kinds of data are blocked.

Various ways could express the above-mentioned difference. One example is different font colors for different types of data, for example, green for blood pressure, yellow for temperature, weight and noise level that are blocked, and red for any data with indicator “Y” which represents the data is missing at the end of user side. Another example is different background colors for different types of data.

By the indicator of showing the data is missing at the user side, the caregivers may not initiate the hopeless request of asking the user to provide the missing data.

Indicating unit 340 could also present the latest un-updated information of the blocked data from the data stored in storage unit 350. And beside the un-updated information, there could be some mechanisms to show how old the un-updated information is, for example, indicating when the un-updated information was received (a field filled with 15:00:00 p.m. Jun. 26, 2005), or indicating how long it is since the un-updated information was received (a field filled with 1:15 hours).

The heaviness of the color may also be used to show how old the un-updated information is. For example, light yellow shows it is less than 1 hour old, medium yellow shows it is less than 2 hours old, and heavy yellow shows it is more than 2 hours old.

Since different types of data may have different time sensitivity level, for example, Weight will typically not change quickly and so a single weight measurement will be valid for a number of days, whereas blood pressure may change more quickly. Thus weight is with low time sensitivity level, and blood pressure is with high time sensitivity level.

The time sensitivity level also may varies with different caring purpose, for example, the time sensitivity of the same data may vary with different diseases. Blood pressure and heart beat rate are more time sensitive for cardiac diseases, but less time sensitive for leg impairment.

In some circumstances, for example, an emergency, a caregiver may still try to exploit the possibility of using the un-updated information. By knowing how old the un-updated (blocked) information is, the caregiver may judge the value of the un-updated information in his/her hand based on his/her professional expertise.

Requesting unit 330 sends a request for demanding the blocked data from a source of the healthcare data. By knowing the data is indicated as “blocked”, for example, body temperature, the caregivers knows the data is available in the user's side, therefore, the caregivers may initiate a request of asking the user to provide the needed data.

It is understandable requesting unit 330 could also send some information to the user's side to override the controlling classification if needed.

The desire of the invention can also be implemented by means of a suitably programmed computer provided with a computer program for monitoring healthcare data. The computer program product for monitoring healthcare data, comprises code for receiving a part of the healthcare data and an indication(s) of the blocking status of the rest of the healthcare data, code for presenting the healthcare data, and code for indicating the blocking status of the rest of the healthcare data according to the indication.

Such a computer program product may be stored in a storage carrier.

These portions of program code may be provided to a processor to produce a machine, such that the code that executes on the processor create means for implementing the functions specified as above.

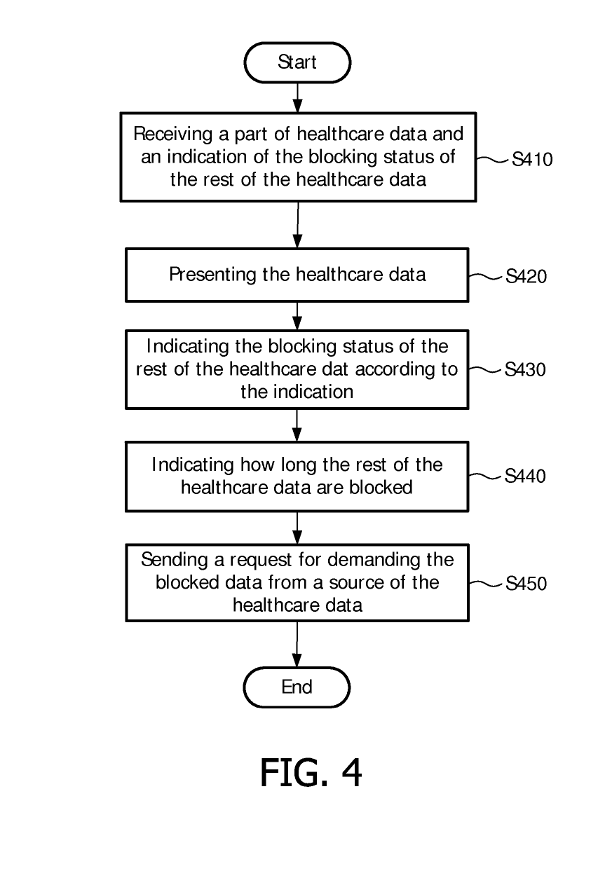

FIG. 4 is a flow chart illustrating a healthcare data monitoring process according to another embodiment of the invention. This process could be implemented on monitoring apparatus 300.

Firstly, receiving a part of healthcare data (blood pressure) and an indication(s) of the blocking status of the rest of the healthcare data (“X” marks for body temperature, weight and noise level). (step S410).

Secondly, presenting the healthcare data (step S420). In this embodiment, the data of blood pressure is presented on a display.

Thirdly, indicating the blocking status of the rest of the healthcare data according to the indication. (Step S430).

In this embodiment, the received data of body temperature, weight and noise level are “X” marks, accordingly the fields of body temperature, weight and noise level on the display are indicated in a different presenting way from the normal presenting way of presenting unit 320 to show the three kinds of data are blocked, for example, green for blood pressure, yellow for temperature, weight and noise level that are blocked,

Fourthly, indicating how long the rest of the healthcare data is blocked. (Step S440).

Beside the un-updated (blocked) information, there could be some fields to show how old the un-updated information is, for example, a field filled with 1:15 hours indicating one hour and a quarter has passed since the un-updated information was received.

Finally, sending a request for demanding the blocked data from a source of the healthcare data if needed. (Step S450).

Since body temperature is indicated as “blocked”, the caregivers knows the data is available in the user's side, therefore, the caregivers may initiate a request of asking the user under his/her wish to provide the body temperature data.

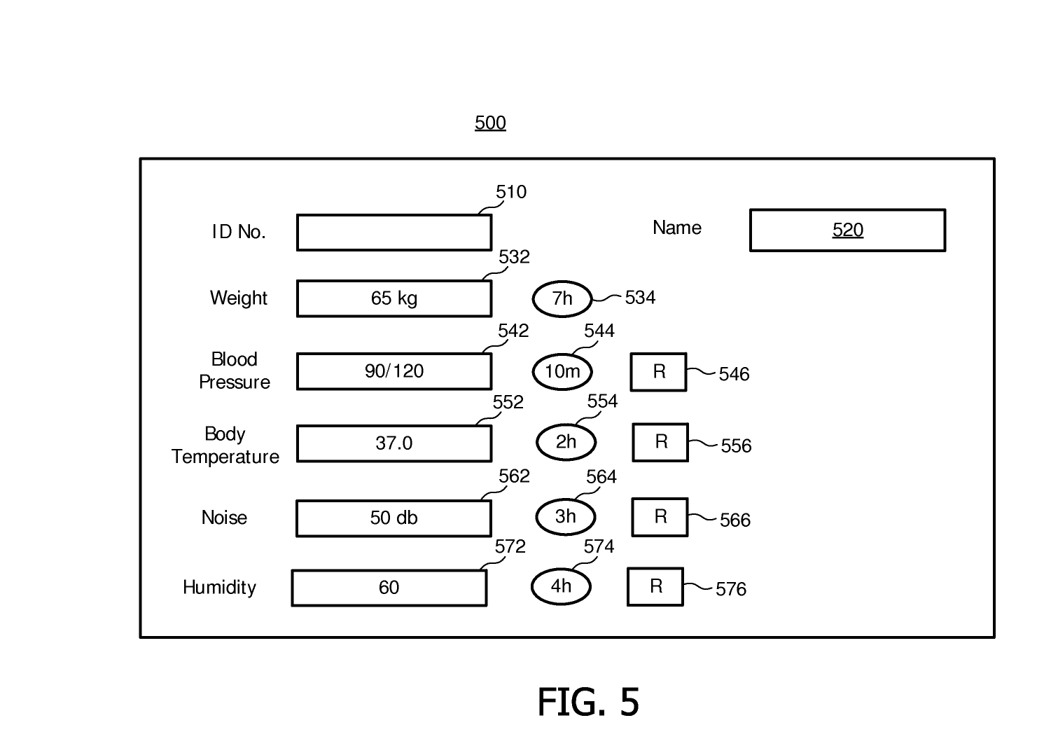

FIG. 5 is a schematic layout of the healthcare-data-monitoring interface according to another embodiment of the invention.

Field 510 represents the identification number of the person being monitored.

Field 520 represents the name of the person being monitored.

Field 532 show “65 Kg” in yellow indicating “weight” is blocked and not updated any more. And field 534 shows “7 h” indicating the data of weight is 7 hours old.

Field 542 show “90/120” in green indicating “blood pressure” is not blocked and is being updated periodically. Field 544 shows “10 m” indicating the data of blood pressure is 10 minutes old. And field 546 show the caregivers could initiate a request for an instant data of the blood pressure.

Field 552 show “37.0” in yellow indicating “body temperature” is blocked and is not updated any more. Field 554 shows “2 h” indicating the data of body temperature is 2 hours old. And field 556 show the caregivers could initiate a request for an instant data of the body temperature.

Field 562 show “50 db” in yellow indicating “noise” is blocked and is not updated any more. Field 564 shows “3 h” indicating the data of noise is 3 hours old. And field 566 show the caregivers could initiate a request for an instant data of the noise.

Field 572 show “60” in red indicating “humidity” is missing at the user side. Field 574 shows “4 h” indicating the data of humidity is 4 hours old. And field 576 is deactivated in gray shows the caregivers could not initiate a request for an instant data of the humidity.

While the invention has been described in conjunction with specific embodiments, it is evident that many alternatives, modifications and variations will be apparent to those skilled in the art in light of the foregoing description. Accordingly, it is intended to embrace all such alternatives, modifications and variations as fall within the spirit and scope of the appended claims.

1. An apparatus for controlling healthcare data, comprising:
receiving means for receiving the healthcare data,
obtaining means for obtaining a user input for controlling dispatch of the healthcare data, and transmitting means for transmitting a part of the healthcare data and an indication(s) of blocking status of a remaining part of the healthcare data to an outside source, according to the user input.

2. The apparatus of claim 1, further comprising capturing means for gathering the healthcare data.

3. The apparatus of claim 2, wherein the gathering means include a biosensor.

4. The apparatus of claim 1, wherein the healthcare data includes life data.

5. The apparatus of claim 1, wherein the healthcare data includes life related environmental data.

6. An interface for monitoring healthcare data, comprising:
receiving means for receiving a part of the healthcare data and an indication(s) of a blocking status of a remaining part of the healthcare data,
presenting means for presenting the healthcare data, and
indicating means for indicating the blocking status of the remaining part of the healthcare data according to the indication.

7. The interface of claim 6, further comprising requesting means for sending a request for demanding blocked data from a source of the healthcare data.

8. The interface of claim 6, wherein the indicating means further indicates how long the remaining part of the healthcare data is blocked.

9. The interface of claim 6, wherein the healthcare data includes life data.

10. The interface of claim 6, wherein the healthcare data includes life related environmental data.

11. A method for controlling healthcare data, comprising:
receiving the healthcare data
obtaining an user input for controlling dispatch of the healthcare data,
transmitting a part of the healthcare data and an indication(s) of blocking status of a remaining part of the healthcare data to an outside source according to the user input.

12. A method for monitoring healthcare data, comprising:
receiving a part of the healthcare data and an indication(s) of blocking status of a remaining part of the healthcare data,
presenting the healthcare data, and
indicating the blocking status of the remaining part of the healthcare data according to the indication.

13. The method of claim 12, further comprising the step of sending a request for demanding blocked data from a source of the healthcare data.

14. The method of claim 12, further comprising the step of indicating how long the remaining part of the healthcare data is blocked.

15-16. (canceled)
